(19) United States
(12) Patent Application Publication
(10) Pub. No.: US 2016/0046899 A1
(43) Pub. Date: Feb. 18, 2016

(54) REACTOR WITH INTEGRATED ILLUMINATION

(71) Applicants: FERMENTALG, Libourne (FR); PIERRE GUERIN, Mauze Sur Le Mignon (FR)

(72) Inventors: Patrice GARNIER, LAGORCE (FR); Francois GODART, VAYRES (FR); Pierre CALLEJA, LIBOURNE (FR); Samuel GIRAUD, LA ROCHELLE (FR); Doru FELEZEU, PARIS (FR)

(21) Appl. No.: 14/784,241

(22) PCT Filed: Apr. 11, 2014
(86) PCT No.: PCT/FR2014/050891

§ 371 Date: Oct. 13, 2015

(30) Foreign Application Priority Data
Apr. 22, 2013 (FR) 1353641

(51) Int. Cl. C12M 1/00; C12M 1/06

(52) U.S. Cl. CPC C12M 31/10; C12M 21/02; C12M 27/02

(57) ABSTRACT

A reactor includes a tank intended to contain a mass to be treated, for example formed from microorganisms, an assembly that rotates about an axis to ensure stirring of this mass, and a plurality of illumination sources to assist the treatment of this mass. The tank has an inner wall with plates fixed thereto, whose planes are oriented towards the axis of the rotating assembly and parallel to same, so as to prevent the formation of a vortex within the mass under the action of the rotating assembly; the illumination sources are carried by the plates, being encapsulated, with at least the part of the plates that carries them, in a material compatible with the mass treated and in a thickness that allows light to be diffused into the tank. This configuration may be scaled, while keeping the ratio of the illumination surface relative to the tank volume approximately constant.

The invention concerns a reactor with integrated illumination, in particular configured for the culture of photosensitive microorganisms. It may be a bioreactor but also a chemical or physico-chemical reactor.

The concept of bioreactor, or biological reactor, here designates a reactor within which biological phenomena develop, such as the growth of cultures of pure microorganisms or of a consortium of microorganisms (in particular microalgae), in very diverse fields such as the treatment of effluents, the production of biomass containing valuable biomolecules (that is to say biomolecules which it is known how to exploit). This concept thus encompasses among others the reactors called fermentors.

A reactor typically comprises a closed tank within which is mounted a mixing or agitating member configured to promote homogenization of the content of the tank; such a mixing member is usually constituted by a vertical shaft carrying blades or turbines, the movement of which, within the mass being treated in the reactor, provides the mixing and its homogeneity.

Various types of operating conditions may be necessary for the growth of biological species inside such a bioreactor or fermentor; thus, in particular, growth regimes are known which are heterotrophic (with provision of a source of carbon such as sugars), autotrophic (or photoautotrophic) with provision of light (the term photosynthesis is also used) or mixotrophic (with provision of a source of carbon and a source of light). A scientific approach to this subject is addressed in particular in the document “Astaxanthin production by Haematococcus pluvialis under illumination with LEDs” by Katsuda et al, which appeared in Enzyme and Microbial Technology 35 (2004) 81-86, or in the document “Effects of using light-emitting diodes on the cultivation of Spirulina platensis” by Wang et al, which appeared in the Biochemical Engineering Journal 37 (2007) 21-25.

Various configurations have already been proposed to implement such a provision of light, continuously or cyclically (with cycles the length of which may vary from a few minutes to several hours), or even in the form of pulses in a flash-like manner, with a spectrum approaching that of daylight or on the contrary narrowly centered on a chosen wavelength. Reactors adapted to such a provision of light are sometimes called photoreactors or photobioreactors (when they are the site of biological treatments).

A well-mastered configuration consists of providing the tank with windows, for example between six and twelve according to the size of the tank, enabling the penetration of light generated from outside the tank. Such windows may be of 5 to 20 cm diameter in practice. A drawback of such a configuration is that the windows limit the surface area of illumination and absorb or reflect a significant portion of the photons emitted by the light source.

For this reason, other configurations have been proposed in which light sources are implanted within the tank.

Thus, document WO-2002/086053, after having mentioned the case of tanks provided with windows, describes an assembly comprising a growth tank, an illumination tank in communication with the growth tank and provided with a light source, preferably situated inside that illumination tank, with a system for circulating microorganisms from one tank to the other; the illumination tank is in practice tubular and comprises deflectors to ensure that the flow of the microorganisms is turbulent; the light source is preferably concentric with the illumination tank and may be constituted by a metal halide lamp, for example a high pressure sodium lamp. It is recommended for the growth tank to further comprise a light source, providing illumination through windows comprised by the tank wall or formed from glass tubes immersed in the microorganism mass. The document US-2010/0190227 (derived from international patent application WO-2008/145719) concerns a bioreactor comprising parts made of molded plastic material in which light emitting diodes (LEDs for short) are integrated; it is envisioned for the parts to be plates or strips when the inside walls of the tank are to be equipped, but it is recommended for those parts to be tubular, preferably in bundles, when those light sources are disposed within the reactor, as is shown by the examples considered, in which case the diodes receive power through the tank cover. The LED diodes emit in the red range, for example. The plastic material in which they are implanted may be silicone.

Furthermore, the document U.S. Pat. No. 7,824,904 describes a reactor in which the mixing device comprises mixing blades carrying light sources such as lamps, bulbs or LEDs.

As regards WO-2011/154886, this describes the same reactor comprising an illumination system joined to a turning assembly.

These various documents show that the fact of generating light outside the tank and making it enter the tank is generally considered as being insufficiently effective and that it is very widely considered that good efficacy requires light sources to be disposed within the actual mass contained in the tank, preferably on the turning assembly disposed therein; various solutions are proposed for the connection of those sources to the outside.

It is worth noting that the utility of illumination within a reactor is not limited to biological treatments; thus, UV radiation may be useful for sterilizing media to be treated (see, for example, the document U.S. Pat. No. 4,456,512); furthermore there are cases wherein light radiation, which may or may not be visible, can promote chemical reactions or physico-chemical treatments.

The invention is directed to a reactor in the tank of which light sources are disposed so as to enable effective illumination of a mass to treat contained in the tank while minimizing the modifications made to the tank for the integration of those light sources.

To that end, the invention provides a reactor comprising a tank configured to contain a mass to treat and provided with an assembly turning around an axis configured to provide mixing of that mass to treat and further provided with a plurality of illumination sources configured to promote the treatment of that mass, said tank having an inside wall to which are fastened plates the planes of which are oriented towards the rotational axis of the turning assembly and parallel thereto, so as to prevent the formation of a vortex within the mass to treat under the action of the turning assembly, said illumination sources being carried by said plates and being encapsulated, with at least the part of those plates which carries them, in a material compatible with the mass to treat and having a thickness enabling said light to be diffused towards the inside of the tank.

It can be understood that the mass to be treated is liquid, pasty or particulate, that is to say, having sufficient fluidity to be able to be mixed. It may be a microorganism mass for which, for example, it is desired to promote the growth and/or the enrichment; but this mass may also be a very fluid mass, or on the contrary granular, for which it is desired, for example, to provide good sterilization.

Thus, the invention takes advantage of the fact that a certain number of reactor tanks comprise plates extending from their inside wall towards the center of the tank, while remaining at a distance from the turning assembly (the tanks are usually cylindrical such that these plates are radially orientated); such plates are often called baffles, and are configured to avoid the appearance of vortices inside the tank when very turbulent mixing is performed by the turning assembly, which is usually provided with mixing blades. The fact that the light sources are carried by such plates enables a much better SN ratio (ratio of illumination surface area to the tank volume) than the known implemented solutions.

Thus, the configuration of the invention enables better illumination efficacy relative to a configuration in which the light enters by windows from sources located outside; moreover, the baffles give a much larger support area than in the configurations in which the illumination sources are located on the turning assembly or on tubular parts immersed in the mass to treat, or on the bottom of the tank or roof of the tank. Furthermore, the configuration of the invention allows one to obtain an SN ratio the order of magnitude of which may be maintained when the size of the tank is increased; thus, if the height and two transverse dimensions of the tank are increased in a ratio R, and if the ratio between the transverse dimension of the plates and the transverse dimensions of the tank and that between the height of those plates and the height of the tanks, the SN ratio becomes SNR (a doubling in the volume of the tank only leads to a drop of 20% of the SN ratio; conversely, it suffices to increase the ratio between the transverse dimension of the plates and that of the tank by 20% to maintain the SN ratio while doubling the volume of the tank); by varying the number of plates carrying the illumination sources, it may be noted that if the volume of a tank is increased tenfold, the SN ratio may be maintained at least approximately by merely doubling the number of plates while maintaining the ratio between the dimensions of those plates and those of the tank. Based on a given tank, a tank designer can thus quite easily extrapolate the dimensions to give to a tank of larger dimensions to obtain a given level of performance. The configuration of the invention, thus, has the advantage of facilitating the design of increasingly large tanks based on smaller tanks, for example, tanks having served as prototypes.

It should be noted that the fact that the illumination sources are situated near the inside wall of the tank (since they extend along it by one edge) simplifies the connection of the sources with the outside, compared with the case of an implantation within the mass to treat, for example, on the turning assembly.

The fact that the illumination sources are carried by plates which are static relative to the tank avoids modifying the space occupied by the turning members, their inertia or the power necessary for their movement.

In fact, it has appeared that, contrary to the recommendations of various prior documents, the fact of disposing the illumination sources away from the central portion of the tank makes it possible to obtain a high illumination power per unit volume without having to use very powerful illumination sources; It suffices to implant a sufficient number of illumination sources, even if their individual power is moderate; the configuration of the invention thus enables good illumination for a lower installation cost than in the known solutions. In practice, the mixing provided by the turning assembly suffices to ensure that the whole of the mass to treat benefits from the illumination. However, the fact that the illumination sources are disposed on the baffles and not on the inside wall of the tank optimizes the fraction of the mass to treat which, during the mixing, comes close to those illumination sources.

Another advantage of disposing the illumination sources on the plates that serve as baffles is to maximize the available surface area for carrying such illumination sources; indeed, the plurality of illumination sources may comprise illumination sources distributed on each of the faces of said plates.

The plates carrying the illumination sources may be fixed.

However, advantageously, the plates carrying the illumination sources may be demountable from the wall of the tank, thus they may be able to be extracted from the tank. An advantage is that the maintenance operations for these plates and for these illumination sources, as well as those of the tank, are, thus, simplified, relative to a configuration in which these sources are carried by the inside wall. Another advantage is that the same tank may be equipped with various illumination configurations (it suffices to have several sets of baffles comprising a different number and/or a different nature of individual illumination sources); the fact of disposing of several sets of baffles, whether or not comprising different illumination configurations, furthermore, makes it possible to minimize the operation interruptions during maintenance operations, because one set of baffles may be under maintenance while another set is in service; similarly, in case of failure, the repair may be made easily, possibly after merely replacing the plates by other plates provided with a similar plurality of illumination sources.

Preferably, the illumination sources are light-emitting diodes (LEDs for short); they are sources of light which are well-understood, both in terms of their implementation and their applications. Such sources may have very diverse emission spectra, because white LEDs (simulating sunlight) exist, but also, LEDs with a reduced spectral range (for example, centered on a red, blue or green light). Such illumination sources generate less heat than bulbs or the like; furthermore they have sufficiently small dimensions to be implanted on the faces of the baffles without giving rise to excess thickness, detrimental to the main function of these plates.

The spectrum of the illumination sources is advantageously in the visible range, but, if need be, may also be outside this visible range, for example, in the UV range (for example for applications involving sterilization), or in the infra-red range (for example, in applications directed to generating heat within the mass to treat).

The illumination sources may have very varied control regimes (the term operating regimes may also be used).

Thus, the plurality of illumination sources may be composed of several sub-assemblies, it being possible for the illumination sources of each of those sub-assemblies to have a specific emission spectrum. Additionally or alternatively, the plurality of illumination sources is formed from several sub-assemblies each having the same excitation regime, whether continuous or cyclical, with a constant or variable intensity. Additionally to one or other of the previously mentioned options, or alternatively, the plurality of illumination sources is connected to an operating assembly, such that at least some of the illumination sources may be operated in lighting or non-lighting mode (i.e. that they are switched on or off depending on the moment), in continuous regime, in flash regime or cyclically, and with emission spectra.

As stated, the tank may, as is known per se, be cylindrical and thus have a given diameter; in this case, it is advantageous for the plates carrying the illumination sources to have a radial dimension comprised between 5% and 20% of that diameter, preferably between 7.5% and 15%, for example 10%.

The material in which the illumination sources are encapsulated is chosen according to the technological constraints one wishes to comply with. Thus, it is preferably a thermoplastic material having good transmission properties for both light and heat, in addition to its compatibility with the mass to treat. Thus, this material enables good evacuation of the heat generated by the illumination sources to the mass to treat which thereby acts as a heat sink; indeed, it is advantageous for the encapsulation material to be capable of evacuating, into the mass to treat, the heat generated by the illumination sources, even when they are LED light sources. Alternatively, the circuits for evacuation of heat (produced by the LEDs, or produced by an exothermic process in the reactor) may be provided within the thickness of the baffles. It is furthermore recommended that such a material should be able to withstand high temperatures, between 100° C. and 150° C., such as those that may be used during the sterilization or decontamination of the tank. Such sterilization or decontamination operations may be carried out on the plates or baffles independently of the tank when these plates or baffles are removable.

When the mass to be treated is formed from microorganisms, the material for encapsulation of the illumination sources is preferably totally inert relative to the product without salting out, so as to avoid any risk of contamination of the mass by that encapsulation material.

A particularly suitable material is polysulfone, which combines good compatibility with food standards (including the United States standards of the Food Drug Administration, or FDA for short), a good heat conductivity coefficient (enabling evacuation of heat to the microorganism mass and a semi-transparent character, enabling good light transmission if the material has a thickness chosen between 1 mm and 5 cm; furthermore this material maintains its properties after potential heat treatment for sterilization or cleaning with detergents or acid. If the combination of desired features is modified, other materials may be chosen, for example polyurethane, polypropylene an acrylic material or a polycarbonate.

In practice, the number of plates carrying the plurality of illumination sources is preferably comprised between 4 and 10, which are regularly distributed around the turning assembly; however a smaller number of plates may be in service at a given time.

Objects, features and advantages of the invention appear from the following description, given by way of illustrative non-limiting example, with reference to the accompanying drawing in which:

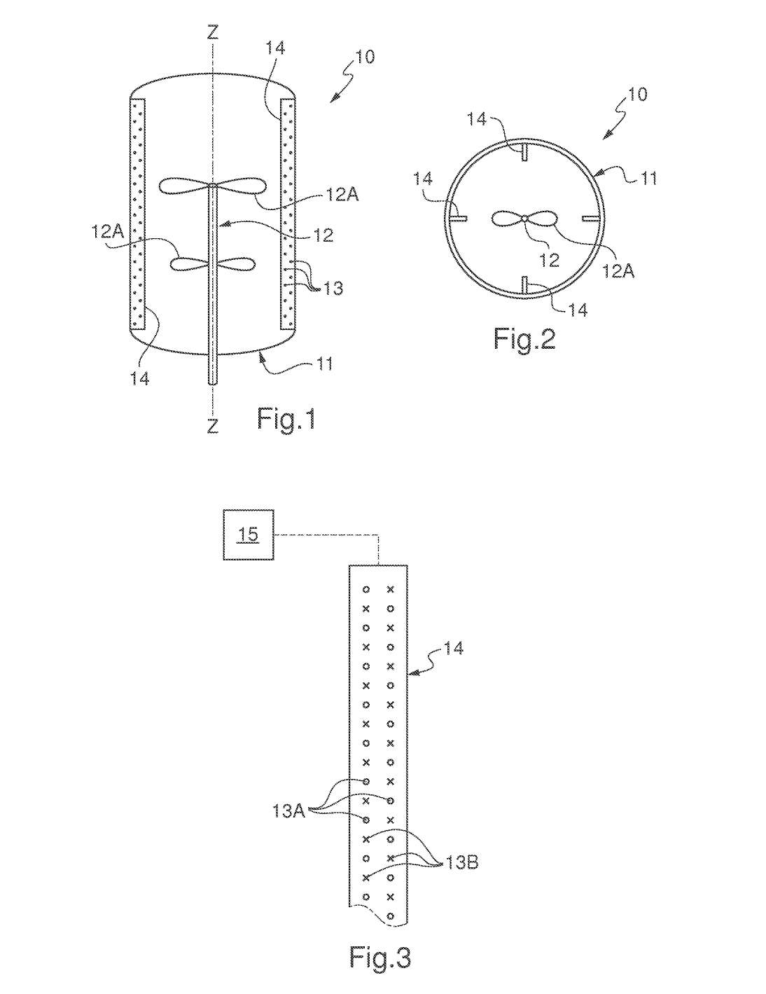

FIG. 1 is a diagram in axial cross-section of a reactor in accordance with the invention,

FIG. 2 is a diagram of that reactor in transverse cross-section, and

FIG. 3 is a detailed view of an example of a plate comprising a plurality of illumination sources.

FIGS. 1 and 2 are diagrammatic representations of a reactor in accordance with the invention; it is described here in relation to the treatment of a mass to treat constituted by a biomass formed from microorganisms, for example, microalgae; it is to be understood however that the following description also applies to other types of reactors, chemical or physico-chemical. This bioreactor, or fermentor, is denoted 10 overall; it mainly comprises a tank 11 configured to contain a mass of microorganisms (not shown), an assembly 12 turning around an axis Z-Z configured to provide mixing of that mass of microorganisms and a plurality of illumination sources 13 configured to promote the growth of that mass of microorganisms; that tank 11 has an inside wall to which are fastened plates 14 the planes of which are oriented towards the axis of the turning assembly and parallel thereto so as to prevent the formation of a vortex within the mass of microorganisms under the action of the turning assembly; such plates 14 are commonly called baffles, as distinct from the mixing blades 12A which the turning member usually comprises.

Such a reactor may, according to its application, comprise other members (not shown), in particular such as an inlet passage for a product to treat or to degrade by means of the biomass, or a supply passage for reagent or nutritive ingredients such as sugars for the proliferation of the biomass, an inlet and outlet passage for air or gas or a passage for drawing off microorganisms.

The illumination sources 13 are carried by said plates and are encapsulated, with at least the part of those plates carrying them, in a material (not shown) compatible with the microorganisms and of a thickness enabling said light to be diffused towards the inside of the tank. Preferably, the illumination sources distributed over a face of such a plate are encapsulated by a material covering the entirety of that face.

By way of example, FIG. 3 represents a detail of such a plate 14. There can be seen therein a first set of illumination sources 13A schematized in the form of small circles and disposed in two columns such that a source of one column is disposed at an intermediate level between those of the closest sources in the other column. There may of course be a greater number of columns, and the sources may be disposed in staggered arrangement so as to form rectangular groups.

In the example represented, a second set of illumination sources 13B can furthermore be seen, which are schematized in the form or small crosses, disposed in a similar configuration to that of the first set, the sources of this second assembly are here disposed so as to alternate with the sources of the first assembly, that is to say that they are disposed in two superposed columns, perpendicularly to the plane of FIG. 3, the sources of a column of one set alternating with the sources of the corresponding column of the other set. As a variant, the columns of the second set may alternate with the columns of the other set and may be more than two in number, possibly different from the number of columns of the first set.

The illumination sources are distributed here over both faces of the plates, the sources 13A being situated on one of the faces while the sources 13B are situated on the opposite face.

As a matter of fact, according to the illumination needs, there may be sources on one side only of a given plate, or on the contrary sources distributed on both faces of such a plate. The sources situated on one face may be distributed on the basis of the distribution of the sources on the other face, in a superposed arrangement or, on the contrary, so as to complement each other; there may also be sources disposed on both faces independently of each other.

When there are illumination sources on both faces of the plate, the encapsulation material advantageously covers the plate on both faces, which promotes good holding of the material onto the plate. For the same reason of good mechanical holding, it can be understood that, even when the sources are only disposed on one face, it may be advantageous for the material to cover not only the face carrying the sources, but also the face that remains bare.

Preferably, the illumination sources are light-emitting diodes, also called LEDs for short. Such sources in particular have small bulk and low electricity consumption while generating a small quantity of heat, relative to the other known illumination sources; when organic LEDs (also called OLEDs) are used, their thickness may be very small. As a variant, these illumination sources may be constituted by the ends of groups of optic fibers extending from one (or more) source(s) disposed outside the tank.

The illumination sources may be all identical in having the same excitation regime; it is however to be understood that, as a variant, the illumination sources contained in the tank may be distributed into several sub-assemblies, each sub-assembly being able to contain a single illumination source or several illumination sources advantageously distributed uniformly, each sub-assembly being individually operated according to several operating parameters: all-or-nothing lighting, or continuous lighting with an intensity that may vary, It may however be simpler to attribute a specific emission spectrum to each sub-assembly, for example one sub-assembly emitting white light, another sub-assembly emitting in the blue range, another sub-assembly emitting in the red range, etc.; according to need, the operator may then choose to excite only the sub-assemblies which are appropriate for the desired illumination regime.

In practice, it is however advantageous to use all the available sources to obtain sufficient illumination.

The main possible excitation regimes of the illumination sources are:
a flash regime at a frequency which may, for example, range from 1 to 150 kHz,
a continuous regime, possibly having slow variations so as to simulate natural illumination).

Whatever the regime, if there are variations, it is possible to provide cycles ranging from 1 s to 24 h. These various regimes consist of providing power to the sources by controlling their excitation, at a level chosen according to need, between 0% and 100% of their nominal power.

These sources are in practice chosen to have an overall illumination power comprised between 1 and 3000 micro-Einstein. Such a range makes it possible to encompass chemical treatments and sterilization treatments.

It can be understood that in the case of bioreactors for the growth of microalgae, it is thus possible, according to needs, to simulate, in particular, one or other of the regimes that are autotrophic, or mixotrophic.

Thus, it is possible to have a homogenous division into the types of illumination sources, or, on the contrary several divisions of the plurality of the illumination sources, which may possibly be combined.

According to a first example, the plurality of illumination sources may be composed of several sub-assemblies, the illumination sources of each of these sub-assemblies having a specific emission spectrum with an unchanging or variable intensity.

According to a second example, the plurality of illumination sources is composed of several sub-assemblies each having the same excitation regime, operating continuously or cyclically, with a constant or variable intensity.

In a third example, the plurality of illumination sources is connected to an operating assembly such that at least some of the illumination sources may be operated in lighting or non-lighting mode, in flash regime, continuously, with or without any cycle.

Despite these various possibilities, it is however to be understood that the option of simply having illumination sources of a single type on a plate is of high practical interest.

A reactor in accordance with the invention has high flexibility of operation and is thus highly polyvalent.

The baffles equipped with illumination sources are advantageously identical so as to be interchangeable; there may however be baffles with illumination sources and baffles without illumination sources.

The density of illumination sources per unit surface area of baffle plate may be chosen according to need (in certain applications, this density may be variable, reduced or even zero at the ends, and greatest at mid-height; as a variant, there are illumination sources only over part of the height of the plates, for example excluding the ends); when these sources are OLEDs, they may be disposed so as to be substantially adjacent; in practice, the illumination sources are distributed regularly with a step size which may be chosen with great freedom, in the range from just a few millimeters (for example 0.5 cm) to the order of a meter (in height and/or in width).

The operation of a reactor involves other operating parameters such as the temperature, the pH, the amount of dissolved oxygen, the mixing speed, the pressure, etc. However, as these parameters are not modified by the configuration of the illumination sources according to the invention, they are not detailed here.

Especially when they are organic LEDs, the sources may be fastened to the baffles simply by bonding.

In a way that is conventional per se, the tank and the baffles are usually of stainless steel or equivalent but other materials may be chosen according to need.

As is known per se, the tank is advantageously cylindrical. If D is its diameter and H its height, the plates carrying the illumination sources preferably have a radial dimension comprised between 7.5% and 15%, for example of the order of 10% (typically between 9% and 11%). This is compatible with the baffle function provided by those plates.

In a way that is particularly advantageous, the plates carrying the illumination sources are demountable from the wall of the tank. This not only gives rise to very easy maintenance, but also great ease for adapting a given tank to a given application, by choosing plates having the right density of illumination sources, or by varying the number of plates carrying such illumination sources.

In the example of FIGS. 1 and 2, the tank is provided with four plates 14 serving as baffles. It is to be understood that this number may be changed; it may be lower (a single baffle already enables at least a partial baffle effect to be provided), or it may even be greater; it is however advantageous to choose it in the range from 2 to 10 (for example 4 to 10) it being specified that this number may be greater with increasing tank diameter to maintain the anti-vortex role. In practice, these plates are regularly distributed around the turning assembly.

Preferably, all the plates serving as baffles are equipped with illumination sources, but it is possible to provide for only some of the baffle plates to serve as a support for illumination sources; this depends on the uses.

The material in which the illumination sources are encapsulated serves to protect the sources relative to the microorganisms which the tank may contain, while promoting the attachment of those sources to their support plate over time. The choice of this material depends on the possible uses of the tank considered; generally, this material must be compatible with the microorganisms while enabling good outward diffusion of the light generated by the sources and, preferably, good diffusion of heat; it is to be understood however that this capacity depends not only on the material but also on the thickness of material that covers the sources (provided that the encapsulation role can be obtained with a small thickness, it is not necessary for the material to be intrinsically transparent; it may be only partially transparent).

In the case of the fermentation of microalgae, it has turned out that polysulfone is a particularly appropriate material, being compatible with microalgae in terms of food standards, being semi-transparent, and being capable of withstanding treatments of sterilization at high temperature and cleaning with detergents and acid; it is however to be understood that other materials may be chosen according to the properties sought.

It is worth noting that, by playing on the two parameters that are the width of the baffles relative to the diameter of the tank and the number of these baffles, it is possible to obtain an illumination surface area per unit volume which remains substantially constant within a wide volume range, as shown by the following table, in which:
“V” designates the useful volume of the tank (in m³),
“H” designates the height of the shell ring (in m),
“Di” designates the inside diameter of the tank (in m),
“CP” designates the baffles,
“CP/Di” designates the ratio between the radial dimension of the baffles and the diameter of the tank,
“S(CP)” designates the total surface area of both faces of a baffle (in m^{2),}
“N (CP)” designates the number of baffles equipped with illumination sources,
“Stot” designates the total surface area of the baffles (in m²)
“Scp/V” designates the surface area of illumination per unit volume of the tank (in m²/m³).

It is to be noted that, by allowing the CP/Di ratio to vary between 7% and 13% and by varying the number of baffles equipped with illumination sources between 1 (for prototype tanks) and 10, the illumination surface area per unit volume may be kept in the range of 0.75 m²/m³+/−0.05 m²/m³.

| | Example of a tank with a H/Di ratio of 2.0 | | | | | | | | | | |
|---|---|---|---|---|---|---|---|---|---|---|---|
| V (m3) | 0.1 | 0.2 | 0.5 | 1 | 2 | 5 | 10 | 15 | 20 | 50 | 100 |
| H (m) | 0.975 | 1.215 | 1.618 | 2.029 | 2.551 | 3.408 | 4.265 | 4.851 | 5.33 | 7.188 | 9.031 |
| Di (m) | 0.404 | 0.509 | 0.69 | 0.89 | 1.097 | 1.489 | 1.874 | 2.147 | 2.362 | 3.204 | 4.034 |
| CP/Di | 0.095 | 0.12 | 0.09 | 0.1 | 0.09 | 0.12 | 0.12 | 0.11 | 0.1 | 0.1 | 0.13 |
| ratio | | | | | | | | | | | |
| S(CP) | 0.0748 | 0.1484 | 0.2009 | 0.3611 | 0.5037 | 1.2178 | 1.9182 | 2.2913 | 2.5178 | 4.606 | 9.472 |
| N (CP) | 1 | 1 | 2 | 2 | 3 | 3 | 4 | 5 | 6 | 8 | 8 |
| Stot (m2) | 0.0748 | 0.1484 | 0.4019 | 0.722 | 1.5111 | 3.653 | 7.672 | 11.456 | 15.107 | 36.848 | 75.776 |
| Scp/V | 0.748 | 0.742 | 0.804 | 0.722 | 0.756 | 0.731 | 0.767 | 0.764 | 0.755 | 0.737 | 0.758 |
| (m2/m3) | | | | | | | | | | | |

It will be understood that the invention applies to bioreactors and fermentors able to be of great variety.

Thus these bioreactors may be reactors for bio-production, for production of photosynthetic, heterotrophic or mixotrophic microorganism biomass, photocatalysis, for treatment of effluents, for example for waste water treatment.

The microorganisms may contain chloroplasts or not. It has been possible to find for those which do not contain any, that they produce pigments via photoreceptors.

The microorganisms may, in particular, be bacteria, fungi, microalgae, etc.

However, the preceding comments also apply to these reactors for physical or physico-chemical treatments, such as reactors for sterilization, or for decontamination, or, for example, for treatment by light-sensitive catalyst.

The light sources may be LEDs (system with one or more wavelengths), with, for example, blue or red LEDs providing alternating illumination, optical fibers, UV lamps, etc. The illumination spectrum may indeed be outside the visible range.

The tank represented has a vertically disposed axis of symmetry, but it may be understood that the above comments may be generalized in the case of a tank having an inclined axis, it then being possible to replace the concepts of height and width by concepts of longitudinal, or axial, dimension, and of transverse dimension (it is to be recalled that it is possible for the tank not to be cylindrical).

It may be understood that one advantage of the described configuration is that it can be transposed to a different scale while maintaining approximately constant the ratio of the illumination surface area relative to the volume of the tank, the assembly being resistant to operations of in situ cleaning or sterilization when it is a bioreactor or a fermentor.

1. A reactor (10) comprising a tank (11) configured to contain a mass to treat and provided with an assembly (12) turning around an axis (Z-Z) configured to provide mixing of that mass to treat and further provided with a plurality of illumination sources (13, 13A, 13B) configured to promote the treatment of that mass, said tank having an inside wall to which are fastened plates (14) the planes of which are oriented towards the axis of the turning assembly and parallel thereto, so as to prevent the formation of a vortex within the mass to treat under the action of the turning assembly, said illumination sources being carried by said plates and being encapsulated, with at least the part of those plates which carries them, in a material compatible with the mass to treat and having a thickness enabling said light to be diffused towards the inside of the tank.

2. A reactor according to claim 1, wherein the plurality of illumination sources comprises illumination sources (13A, 13B) distributed on each of the faces of said plates.

3. A reactor according to claim 1, wherein the plates (14) carrying the illumination sources are demountable from the wall of the tank.

4. A reactor according to claim 1, wherein the illumination sources (13, 13A, 13B) are light-emitting diodes.

5. A reactor according to claim 1, wherein the plurality of illumination sources is composed of several sub-assemblies, the illumination sources of each of those sub-assemblies having a specific emission spectrum.

6. A reactor according to claim 1, wherein the plurality of illumination sources is connected to an operating assembly such that at least some of the illumination sources may be operated in lighting or non-lighting mode, in flash mode, in continuous mode or cyclically.

7. A reactor according to claim 1, wherein the tank is cylindrical while having a given diameter, the plates carrying the illumination sources having a radial dimension comprised between 5% and 20% of that diameter.

8. A reactor according to claim 1, wherein the material in which the illumination sources are encapsulated is a thermoplastic material chosen so as to enable good evacuation of the heat generated by the illumination sources.

9. A reactor according to claim 1, wherein the thermoplastic material in which the illumination sources are encapsulated is polysulfone.

10. A reactor according to claim 1, wherein plates carrying the plurality of illumination sources are from 1 to 10 in number and, when the number is greater than 2, are regularly distributed around the turning assembly.

11. A reactor according to claim 2, wherein the plates (14) carrying the illumination sources are demountable from the wall of the tank.

12. A reactor according to claim 2, wherein the illumination sources (13, 13A, 13B) are light-emitting diodes.

13. A reactor according to claim 2, wherein the plurality of illumination sources is composed of several sub-assemblies, the illumination sources of each of those sub-assemblies having a specific emission spectrum.

14. A reactor according to claim 2, wherein the plurality of illumination sources is connected to an operating assembly such that at least some of the illumination sources may be operated in lighting or non-lighting mode, in flash mode, in continuous mode or cyclically.

15. A reactor according to claim 2, wherein the tank is cylindrical while having a given diameter, the plates carrying the illumination sources having a radial dimension comprised between 5% and 20% of that diameter.

16. A reactor according to claim 2, wherein the material in which the illumination sources are encapsulated is a thermoplastic material chosen so as to enable good evacuation of the heat generated by the illumination sources.

17. A reactor according to claim 2, wherein the thermoplastic material in which the illumination sources are encapsulated is polysulfone.

18. A reactor according to claim 2, wherein plates carrying the plurality of illumination sources are from 1 to 10 in number and, when the number is greater than 2, are regularly distributed around the turning assembly.
